(19) United States
(12) Patent Application Publication
(10) Pub. No.: US 2026/0162811 A1
(43) Pub. Date: Jun. 11, 2026

(54) METHODS AND SYSTEMS FOR IDENTIFYING AND CONTROLLING INEFFICIENCIES IN PHARMACEUTICAL UTILIZATION

(71) Applicant: Mitchell International, Inc., San Diego, CA (US)

(72) Inventors: Cameron Hannum, San Diego, CA (US); Kristina Graves, San Diego, CA (US); Serena Galindo, San Diego, CA (US); Jonathan Rowell, San Diego, CA (US); Christina Klemm, San Diego, CA (US); Benjamin Gerstner, San Diego, CA (US); Amy Van Cleve, San Diego, CA (US)

(21) Appl. No.: 18/977,432

(22) Filed: Dec. 11, 2024

(51) Int. Cl. G16H 40/20

(52) U.S. Cl. CPC G16H 40/20

(57) ABSTRACT

Embodiments of the disclosure provide systems and methods for identifying and controlling inefficiencies in pharmaceutical utilization. Embodiments identify medications at the National Drug Code level that do not provide clinical value over more cost-effective, yet equally efficacious therapeutic alternatives. Embodiments identify price outliers to generate a list which can be used as an exclusion drug list and insight tool for identifying price outlier medications for appropriate Fraud, Waste and Abuse (FWA) mitigation. Embodiments facilitate the identification of drugs having a higher cost than similar medications within the same category, without a corresponding increase in clinical value.

### FIELD OF THE DISCLOSURE

Embodiments of the present disclosure relate generally to methods and systems for processing of prescriptions and more particularly to identifying and controlling inefficiencies in pharmaceutical utilization.

### BACKGROUND

Worker's compensation and other Pharmacy Benefit Managers (PBMs) providing payment for prescription pharmaceuticals continually monitor for Fraud, Waste, and Abuse (FWA). These sectors are vulnerable to such activities due to the complex nature of billing and reimbursement. For example, even if no fraud is involved, a healthcare provider may write a prescription for a particular drug when an equally effective, lower cost alternative is available. Adding to the difficulty monitoring for FWA, new drugs and other pharmaceuticals are constantly being brought to market. Hence, there is a need for improved methods and systems for identifying and controlling inefficiencies in pharmaceutical utilization.

### BRIEF SUMMARY

Embodiments of the disclosure provide systems and methods for identifying and controlling inefficiencies in pharmaceutical utilization. According to one embodiment, a method for identifying and controlling inefficiencies in pharmaceutical utilization can comprise maintaining a list of pharmaceuticals comprising a plurality of categories. Each category can comprise a plurality of pharmaceuticals, each pharmaceutical can be identified by a code, and each pharmaceutical in the list of pharmaceuticals can comprise an identified inefficiency. The list of pharmaceuticals can further comprise an identified substitute for each pharmaceutical in the list of pharmaceuticals.

Information comprising a code identifying a prescribed pharmaceutical can be received from a pharmacy system and a determination can be made as to whether the prescribed pharmaceutical is an identified inefficiency based on the code identifying a prescribed pharmaceutical and the codes identifying the pharmaceuticals in the list of pharmaceuticals. In response to determining the prescribed pharmaceutical is an identified inefficiency, instructions related to the prescribed pharmaceutical and medically or price-driven recommendations for decision support on the prescribed pharmaceutical based on the identified substitute of the pharmaceutical in the list of pharmaceuticals having a code matching the code identifying the prescribed pharmaceutical can be provided to the pharmacy system. Additionally, or alternatively, recommendations and messages related to the prescribed pharmaceutical based on the identified substitute of the pharmaceutical in the list of pharmaceuticals having a code matching the code identifying the prescribed pharmaceutical can be provided to an adjuster system.

In response to determining the prescribed pharmaceutical is not an identified inefficiency based on the code identifying a prescribed pharmaceutical and the codes identifying the pharmaceuticals in the list of pharmaceuticals, a further determination can be made as to whether the prescribed pharmaceutical comprises an outlier from substitute pharmaceuticals in a same category of pharmaceuticals as the prescribed pharmaceutical. Maintaining the list of pharmaceuticals can comprise adding the prescribed pharmaceuticals to the list of pharmaceuticals in response to determining the prescribed pharmaceutical comprises an outlier from the substitute pharmaceuticals in the same category of pharmaceuticals as the prescribed pharmaceutical. Determining whether the prescribed pharmaceutical comprises an outlier from substitute pharmaceuticals in a same category of pharmaceuticals as the prescribed pharmaceutical can comprise applying an interquartile range method to a price of the prescribed pharmaceutical and prices of the substitute pharmaceuticals in the same category of pharmaceuticals as the prescribed pharmaceutical. Determining whether the prescribed pharmaceutical comprises an outlier from substitute pharmaceuticals in a same category of pharmaceuticals as the prescribed pharmaceutical is further based on a prescribed dosage of the prescribed pharmaceutical.

Maintaining the list of pharmaceuticals can further comprise reading a database of drug attributes for a plurality of pharmaceuticals in a plurality of categories. An available pharmaceutical can be analyzed based on the drug attributes of the plurality of pharmaceuticals in a same category as the available pharmaceutical. A determination can be made, based on analyzing the available pharmaceutical, whether the available pharmaceutical comprises a substitute pharmaceutical for at least one pharmaceutical of the plurality of pharmaceuticals in the same category as the available pharmaceutical. The list of pharmaceuticals can then be updated based on determining the available pharmaceutical comprises a substitute pharmaceutical for the at least one pharmaceutical of the plurality of pharmaceuticals in the same category as the available pharmaceutical.

The database of drug attributes can further comprise information identifying pharmaceutical compound kits, combinations packs, private label topical analgesics, prescription topicals, fixed combinations, oral agents and test supplies. In such cases, determining whether the prescribed pharmaceutical is an identified inefficiency can further comprise determining whether the prescribed pharmaceutical is part of a pharmaceutical compound kits, combinations packs, private label topical analgesics, prescription topicals, fixed combinations, oral agents or test supplies pack based on the information identifying pharmaceutical compound kits, combinations packs, private label topical analgesics, prescription topicals, fixed combinations, oral agents and test supplies from the database of drug attributes.

According to another embodiment, a system can comprise a processor and a memory coupled with and readable by the processor. The memory can store therein a set of instructions which, when executed by the processor, causes the processor to maintain a list of pharmaceuticals comprising a plurality of categories. Each category can comprise a plurality of pharmaceuticals, each pharmaceutical can be identified by a code, and each pharmaceutical in the list of pharmaceuticals can comprise an identified inefficiency. The list of pharmaceuticals can further comprise an identified substitute for each pharmaceutical in the list of pharmaceuticals.

The instructions can further cause the processor to receive, from a pharmacy system, information comprising a code identifying a prescribed pharmaceutical and determine whether the prescribed pharmaceutical is an identified inefficiency based on the code identifying a prescribed pharmaceutical and the codes identifying the pharmaceuticals in the list of pharmaceuticals. In response to determining the prescribed pharmaceutical is an identified inefficiency, the instructions can further cause the processor to provide, to the pharmacy system, instructions related to the prescribed pharmaceutical and medically or price-driven recommendations for decision support on the prescribed pharmaceutical based on the identified substitute of the pharmaceutical in the list of pharmaceuticals having a code matching the code identifying the prescribed pharmaceutical. Additionally, or alternatively, the instructions can further cause the processor to provide, to an adjuster system, recommendations and messages related to the prescribed pharmaceutical based on the identified substitute of the pharmaceutical in the list of pharmaceuticals having a code matching the code identifying the prescribed pharmaceutical.

The instructions can further cause the processor to, in response to determining the prescribed pharmaceutical is not an identified inefficiency based on the code identifying a prescribed pharmaceutical and the codes identifying the pharmaceuticals in the list of pharmaceuticals, determine whether the prescribed pharmaceutical comprises an outlier from substitute pharmaceuticals in a same category of pharmaceuticals as the prescribed pharmaceutical. Maintaining the list of pharmaceuticals can comprise adding the prescribed pharmaceuticals to the list of pharmaceuticals in response to determining the prescribed pharmaceutical comprises an outlier from the substitute pharmaceuticals in the same category of pharmaceuticals as the prescribed pharmaceutical. Determining whether the prescribed pharmaceutical comprises an outlier from substitute pharmaceuticals in a same category of pharmaceuticals as the prescribed pharmaceutical can comprise applying an interquartile range method to a price of the prescribed pharmaceutical and prices of the substitute pharmaceuticals in the same category of pharmaceuticals as the prescribed pharmaceutical. Determining whether the prescribed pharmaceutical comprises an outlier from substitute pharmaceuticals in a same category of pharmaceuticals as the prescribed pharmaceutical can be further based on a prescribed dosage of the prescribed pharmaceutical.

The instructions can further cause the processor to read a database of drug attributes for a plurality of pharmaceuticals in a plurality of categories. Maintaining the list of pharmaceuticals can further comprise analyzing an available pharmaceutical based on the drug attributes of the plurality of pharmaceuticals in a same category as the available pharmaceutical, determining, based on analyzing the available pharmaceutical, whether the available pharmaceutical comprises a substitute pharmaceutical for at least one pharmaceutical of the plurality of pharmaceuticals in the same category as the available pharmaceutical, and updating the list of pharmaceuticals based on determining the available pharmaceutical comprises a substitute pharmaceutical for the at least one pharmaceutical of the plurality of pharmaceuticals in the same category as the available pharmaceutical. The database of drug attributes can further comprise information identifying pharmaceutical compound kits, combinations packs, private label topical analgesics, prescription topicals, fixed combinations, oral agents and test supplies. In such cases, determining whether the prescribed pharmaceutical is an identified inefficiency can further comprise determining whether the prescribed pharmaceutical is part of a pharmaceutical compound kits, combinations packs, private label topical analgesics, prescription topicals, fixed combinations, oral agents or test supplies pack based on the information identifying pharmaceutical compound kits, combinations packs, private label topical analgesics, prescription topicals, fixed combinations, oral agents and test supplies from the database of drug attributes.

According to yet another embodiment, a non-transitory, computer-readable medium can comprise a set of instructions stored therein which, when executed by a processor, causes the processor to maintain a list of pharmaceuticals comprising a plurality of categories. Each category can comprise a plurality of pharmaceuticals, each pharmaceutical can be identified by a code, and each pharmaceutical in the list of pharmaceuticals can comprise an identified inefficiency. The list of pharmaceuticals can further comprise an identified substitute for each pharmaceutical in the list of pharmaceuticals.

The instructions can further cause the processor to receive, from a pharmacy system, information comprising a code identifying a prescribed pharmaceutical and determine whether the prescribed pharmaceutical is an identified inefficiency based on the code identifying a prescribed pharmaceutical and the codes identifying the pharmaceuticals in the list of pharmaceuticals. In response to determining the prescribed pharmaceutical is an identified inefficiency, the instructions can further cause the processor to provide, to the pharmacy system, instructions related to the prescribed pharmaceutical and medically or price-driven recommendations for decision support on the prescribed pharmaceutical based on the identified substitute of the pharmaceutical in the list of pharmaceuticals having a code matching the code identifying the prescribed pharmaceutical. Additionally, or alternatively, the instructions can further cause the processor to provide, to an adjuster system, recommendations and messages related to the prescribed pharmaceutical based on the identified substitute of the pharmaceutical in the list of pharmaceuticals having a code matching the code identifying the prescribed pharmaceutical.

The instructions can further cause the processor to, in response to determining the prescribed pharmaceutical is not an identified inefficiency based on the code identifying a prescribed pharmaceutical and the codes identifying the pharmaceuticals in the list of pharmaceuticals, determine whether the prescribed pharmaceutical comprises an outlier from substitute pharmaceuticals in a same category of pharmaceuticals as the prescribed pharmaceutical. Maintaining the list of pharmaceuticals can comprise adding the prescribed pharmaceuticals to the list of pharmaceuticals in response to determining the prescribed pharmaceutical comprises an outlier from the substitute pharmaceuticals in the same category of pharmaceuticals as the prescribed pharmaceutical. Determining whether the prescribed pharmaceutical comprises an outlier from substitute pharmaceuticals in a same category of pharmaceuticals as the prescribed pharmaceutical can comprise applying an interquartile range method to a price of the prescribed pharmaceutical and prices of the substitute pharmaceuticals in the same category of pharmaceuticals as the prescribed pharmaceutical. Determining whether the prescribed pharmaceutical comprises an outlier from substitute pharmaceuticals in a same category of pharmaceuticals as the prescribed pharmaceutical can be further based on a prescribed dosage of the prescribed pharmaceutical.

The instructions can further cause the processor to read a database of drug attributes for a plurality of pharmaceuticals in a plurality of categories. Maintaining the list of pharmaceuticals can further comprise analyzing an available pharmaceutical based on the drug attributes of the plurality of pharmaceuticals in a same category as the available pharmaceutical, determining, based on analyzing the available pharmaceutical, whether the available pharmaceutical comprises a substitute pharmaceutical for at least one pharmaceutical of the plurality of pharmaceuticals in the same category as the available pharmaceutical, and updating the list of pharmaceuticals based on determining the available pharmaceutical comprises a substitute pharmaceutical for the at least one pharmaceutical of the plurality of pharmaceuticals in the same category as the available pharmaceutical. The database of drug attributes can further comprise information identifying pharmaceutical compound kits, combinations packs, private label topical analgesics, prescription topicals, fixed combinations, oral agents and test supplies. In such cases, determining whether the prescribed pharmaceutical is an identified inefficiency can further comprise determining whether the prescribed pharmaceutical is part of a pharmaceutical compound kits, combinations packs, private label topical analgesics, prescription topicals, fixed combinations, oral agents or test supplies pack based on the information identifying pharmaceutical compound kits, combinations packs, private label topical analgesics, prescription topicals, fixed combinations, oral agents and test supplies from the database of drug attributes.

### BRIEF DESCRIPTION OF THE DRAWINGS

FIG. 1 is a block diagram illustrating elements of an exemplary computing environment in which embodiments of the present disclosure may be implemented.

FIG. 2 is a block diagram illustrating elements of an exemplary computing device in which embodiments of the present disclosure may be implemented.

FIG. 3 is a block diagram illustrating an exemplary environment for identifying and controlling inefficiencies in pharmaceutical utilization according to one embodiment of the present disclosure.

FIG. 4 is a flowchart illustrating an exemplary process for identifying and controlling inefficiencies in pharmaceutical utilization according to one embodiment of the present disclosure.

FIG. 5 is a flowchart illustrating additional details of an exemplary process for identifying and controlling inefficiencies in pharmaceutical utilization according to one embodiment of the present disclosure.

FIG. 6 is a flowchart illustrating further details of an exemplary process for identifying and controlling inefficiencies in pharmaceutical utilization according to one embodiment of the present disclosure.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

In the following description, for the purposes of explanation, numerous specific details are set forth in order to provide a thorough understanding of various embodiments disclosed herein. It will be apparent, however, to one skilled in the art that various embodiments of the present disclosure may be practiced without some of these specific details. The ensuing description provides exemplary embodiments only and is not intended to limit the scope or applicability of the disclosure. Furthermore, to avoid unnecessarily obscuring the present disclosure, the preceding description omits a number of known structures and devices. This omission is not to be construed as a limitation of the scopes of the claims. Rather, the ensuing description of the exemplary embodiments will provide those skilled in the art with an enabling description for implementing an exemplary embodiment. It should however be appreciated that the present disclosure may be practiced in a variety of ways beyond the specific detail set forth herein.

While the exemplary aspects, embodiments, and/or configurations illustrated herein show the various components of the system collocated, certain components of the system can be located remotely, at distant portions of a distributed network, such as a Local-Area Network (LAN) and/or Wide-Area Network (WAN) such as the Internet, or within a dedicated system. Thus, it should be appreciated, that the components of the system can be combined in to one or more devices or collocated on a particular node of a distributed network, such as an analog and/or digital telecommunications network, a packet-switch network, or a circuit-switched network. It will be appreciated from the following description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, Non-Volatile Random-Access Memory (NVRAM), or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a Compact Disk Read-Only Memory (CD-ROM), any other optical medium, punch cards, paper tape, any other physical medium with patterns of holes, a Random-Access Memory (RAM), a Programmable Read-Only Memory (PROM), and Erasable Programable Read-Only Memory (EPROM), a Flash-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

A “computer readable signal” medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable medium may be transmitted using any appropriate medium, including but not limited to wireless, wireline, optical fiber cable, Radio Frequency (RF), etc., or any suitable combination of the foregoing.

The terms “determine,” “calculate,” and “compute,” and variations thereof, as used herein, are used interchangeably and include any type of methodology, process, mathematical operation or technique.

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the disclosure, brief description of the drawings, detailed description, abstract, and claims themselves.

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Any combination of one or more computer readable medium(s) may be utilized. The computer readable medium may be a computer readable signal medium or a computer readable storage medium.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as Programmable Logic Device (PLD), Programmable Logic Array (PLA), Field Programmable Gate Array (FPGA), Programmable Array Logic (PAL), special purpose computer, any comparable means, or the like. In general, any device(s) or means capable of implementing the methodology illustrated herein can be used to implement the various aspects of this disclosure. Exemplary hardware that can be used for the disclosed embodiments, configurations, and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

Examples of the processors as described herein may include, but are not limited to, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or Very Large-Scale Integration (VLSI) design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or Common Gateway Interface (CGI) script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

Various additional details of embodiments of the present disclosure will be described below with reference to the figures. While the flowcharts will be discussed and illustrated in relation to a particular sequence of events, it should be appreciated that changes, additions, and omissions to this sequence can occur without materially affecting the operation of the disclosed embodiments, configuration, and aspects.

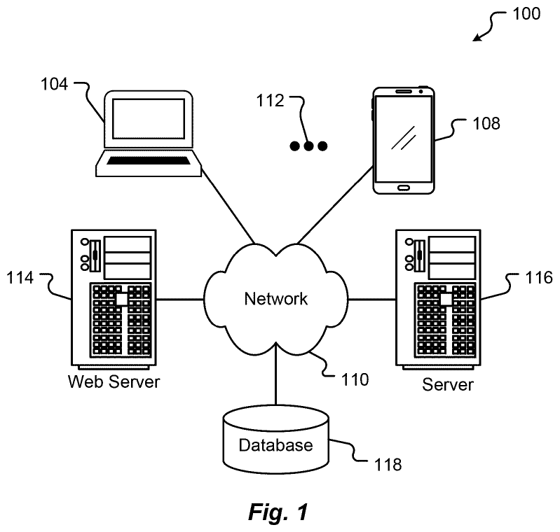

FIG. 1 is a block diagram illustrating elements of an exemplary computing environment in which embodiments of the present disclosure may be implemented. More specifically, this example illustrates a computing environment 100 that may function as the servers, user computers, or other systems provided and described herein. The environment 100 includes one or more user computers, or computing devices, such as a computing device 104, a communication device 108, and/or more 112. The computing devices 104, 108, 112 may include general purpose personal computers (including, merely by way of example, personal computers, and/or laptop computers running various versions of Microsoft Corp.'s Windows® and/or Apple Corp.'s Macintosh® operating systems) and/or workstation computers running any of a variety of commercially-available UNIX® or UNIX-like operating systems. These computing devices 104, 108, 112 may also have any of a variety of applications, including for example, database client and/or server applications, and web browser applications. Alternatively, the computing devices 104, 108, 112 may be any other electronic device, such as a thin-client computer, Internet-enabled mobile telephone, and/or personal digital assistant, capable of communicating via a network 110 and/or displaying and navigating web pages or other types of electronic documents. Although the exemplary computer environment 100 is shown with two computing devices, any number of user computers or computing devices may be supported.

Environment 100 further includes a network 110. The network 110 may can be any type of network familiar to those skilled in the art that can support data communications using any of a variety of commercially-available protocols, including without limitation Session Initiation Protocol (SIP), Transmission Control Protocol/Internet Protocol (TCP/IP), Systems Network Architecture (SNA), Internetwork Packet Exchange (IPX), AppleTalk, and the like. Merely by way of example, the network 110 maybe a Local Area Network (LAN), such as an Ethernet network, a Token-Ring network and/or the like; a wide-area network; a virtual network, including without limitation a Virtual Private Network (VPN); the Internet; an intranet; an extranet; a Public Switched Telephone Network (PSTN); an infra-red network; a wireless network (e.g., a network operating under any of the IEEE 802.9 suite of protocols, the Bluetooth® protocol known in the art, and/or any other wireless protocol); and/or any combination of these and/or other networks.

The system may also include one or more servers 114, 116. In this example, server 114 is shown as a web server and server 116 is shown as an application server. The web server 114, which may be used to process requests for web pages or other electronic documents from computing devices 104, 108, 112. The web server 114 can be running an operating system including any of those discussed above, as well as any commercially-available server operating systems. The web server 114 can also run a variety of server applications, including SIP servers, HyperText Transfer Protocol (secure) (HTTP(s)) servers, FTP servers, CGI servers, database servers, Java servers, and the like. In some instances, the web server 114 may publish operations available operations as one or more web services.

The environment 100 may also include one or more file and or/application servers 116, which can, in addition to an operating system, include one or more applications accessible by a client running on one or more of the computing devices 104, 108, 112. The server(s) 116 and/or 114 may be one or more general purpose computers capable of executing programs or scripts in response to the computing devices 104, 108, 112. As one example, the server 116, 114 may execute one or more web applications. The web application may be implemented as one or more scripts or programs written in any programming language, such as Java™, C, C #®, or C++, and/or any scripting language, such as Perl, Python, or Tool Command Language (TCL), as well as combinations of any programming/scripting languages. The application server(s) 116 may also include database servers, including without limitation those commercially available from Oracle®, Microsoft®, Sybase®, IBM® and the like, which can process requests from database clients running on a computing device 104, 108, 112.

The web pages created by the server 114 and/or 116 may be forwarded to a computing device 104, 108, 112 via a web (file) server 114, 116. Similarly, the web server 114 may be able to receive web page requests, web services invocations, and/or input data from a computing device 104, 108, 112 (e.g., a user computer, etc.) and can forward the web page requests and/or input data to the web (application) server 116. In further embodiments, the server 116 may function as a file server. Although for ease of description, FIG. 1 illustrates a separate web server 114 and file/application server 116, those skilled in the art will recognize that the functions described with respect to servers 114, 116 may be performed by a single server and/or a plurality of specialized servers, depending on implementation-specific needs and parameters. The computer systems 104, 108, 112, web (file) server 114 and/or web (application) server 116 may function as the system, devices, or components described herein.

The environment 100 may also include a database 118. The database 118 may reside in a variety of locations. By way of example, database 118 may reside on a storage medium local to (and/or resident in) one or more of the computers 104, 108, 112, 114, 116. Alternatively, it may be remote from any or all of the computers 104, 108, 112, 114, 116, and in communication (e.g., via the network 110) with one or more of these. The database 118 may reside in a Storage-Area Network (SAN) familiar to those skilled in the art. Similarly, any necessary files for performing the functions attributed to the computers 104, 108, 112, 114, 116 may be stored locally on the respective computer and/or remotely, as appropriate. The database 118 may be a relational database, such as Oracle 20i®, that is adapted to store, update, and retrieve data in response to Structured Query Language (SQL) formatted commands.

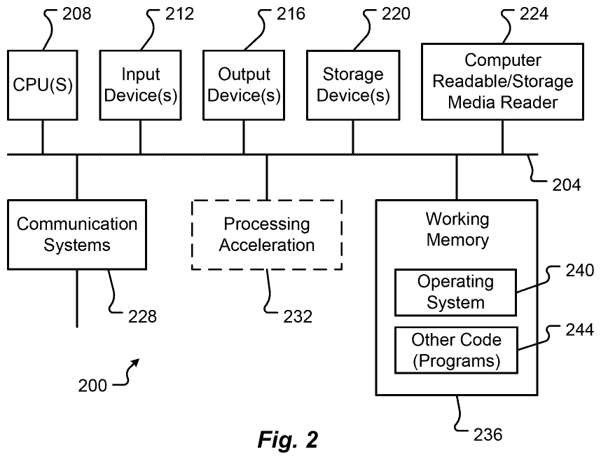

FIG. 2 is a block diagram illustrating elements of an exemplary computing device in which embodiments of the present disclosure may be implemented. More specifically, this example illustrates one embodiment of a computer system 200 upon which the servers, user computers, computing devices, or other systems or components described above may be deployed or executed. The computer system 200 is shown comprising hardware elements that may be electrically coupled via a bus 204. The hardware elements may include one or more Central Processing Units (CPUs) 208; one or more input devices 212 (e.g., a mouse, a keyboard, etc.); and one or more output devices 216 (e.g., a display device, a printer, etc.). The computer system 200 may also include one or more storage devices 220. By way of example, storage device(s) 220 may be disk drives, optical storage devices, solid-state storage devices such as a Random-Access Memory (RAM) and/or a Read-Only Memory (ROM), which can be programmable, flash-updateable and/or the like.

The computer system 200 may additionally include a computer-readable storage media reader 224; a communications system 228 (e.g., a modem, a network card (wireless or wired), an infra-red communication device, etc.); and working memory 236, which may include RAM and ROM devices as described above. The computer system 200 may also include a processing acceleration unit 232, which can include a Digital Signal Processor (DSP), a special-purpose processor, and/or the like.

The computer-readable storage media reader 224 can further be connected to a computer-readable storage medium, together (and, optionally, in combination with storage device(s) 220) comprehensively representing remote, local, fixed, and/or removable storage devices plus storage media for temporarily and/or more permanently containing computer-readable information. The communications system 228 may permit data to be exchanged with a network and/or any other computer described above with respect to the computer environments described herein. Moreover, as disclosed herein, the term “storage medium” may represent one or more devices for storing data, including ROM, RAM, magnetic RAM, core memory, magnetic disk storage mediums, optical storage mediums, flash memory devices and/or other machine-readable mediums for storing information.

The computer system 200 may also comprise software elements, shown as being currently located within a working memory 236, including an operating system 240 and/or other code 244. It should be appreciated that alternate embodiments of a computer system 200 may have numerous variations from that described above. For example, customized hardware might also be used and/or particular elements might be implemented in hardware, software (including portable software, such as applets), or both. Further, connection to other computing devices such as network input/output devices may be employed.

Examples of the processors 208 as described herein may include, but are not limited to, at least one of Qualcomm® Snapdragon® 800 and 801, Qualcomm® Snapdragon® 620 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

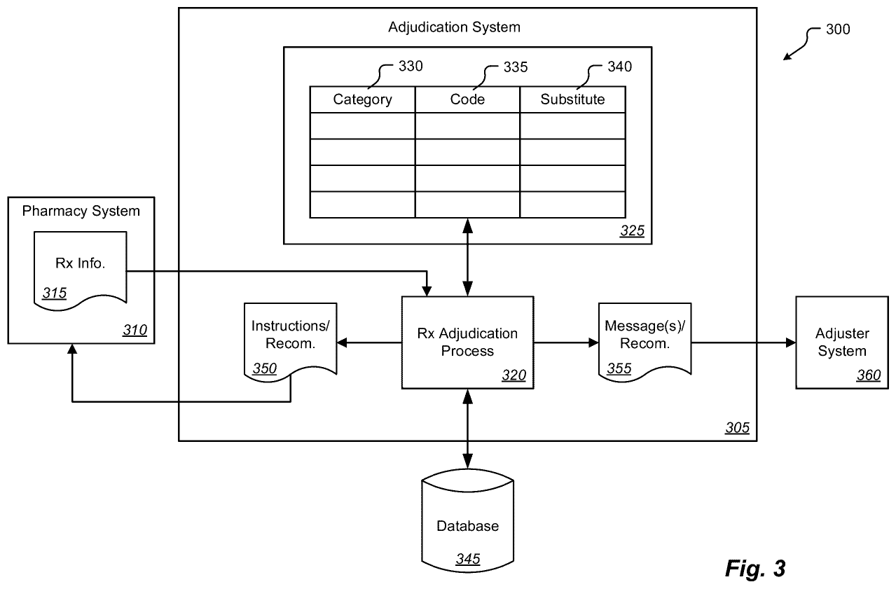

FIG. 3 is a block diagram illustrating an exemplary environment for identifying and controlling inefficiencies in pharmaceutical utilization according to one embodiment of the present disclosure. As illustrated in this example, the environment 300 can comprise an adjudication system 305. The adjudication system 305 can comprise any one or more servers and/or other computing devices as described above. The environment 300 can further comprise a pharmacy system 310 and adjuster system 360. Each of the pharmacy system 310 and adjuster system 360 can also comprise any one or more computing devices as described above. Both the pharmacy system 310 and adjuster system 360 can be coupled with the adjudication system 305 via a communications network (not shown here). The communications network can comprise any one or more wired and/or wireless, local-area and/or wide-area networks as known in the art including, but not limited to, the Internet.

More specifically, the pharmacy system 310 can comprise, for example, a point-of-sale device or other computing device in and used by a pharmacy in the processing of a prescription. When a prescription is received by the pharmacy system 310, information 315 related to that prescription can be sent by the pharmacy system 310, through the communications network (not shown here), to the adjudication system 305 for review. The information 315 related to the prescription can include, but is not limited to, one or more codes identifying one or more prescribed pharmaceuticals. For example, these codes can comprise a National Drug Code (NDC) as known in the art.

The adjudication system 310 can execute a prescription adjudication process 320 for reviewing the prescription information 315 received from the pharmacy system 310. According to one embodiment, the prescription adjudication process 320 can maintain a list 325 of pharmaceuticals. Each pharmaceutical in the list of pharmaceuticals can comprise an identified inefficiency. For each entry in the list 325 the identified pharmaceutical can be identified by one of a plurality of categories. These categories can include, but are not limited to, a pharmaceutical compound kits category, a combinations packs category, a private label topical analgesics category, a prescription topicals category, a fixed combinations category, an oral agents category, a test supplies pack category, and/or others. Each category can comprise a plurality of pharmaceuticals. Each pharmaceutical in the list 325 can be further identified by a code 335, e.g., an NDC, and. The list of pharmaceuticals can further comprise an identified substitute 340 for each pharmaceutical in the list of pharmaceuticals. The substitute can be identified, for example, by a code such as a Generic Product Identifier (GPI). Other information (not shown here) for each entry in the list can include, but is not limited to, a product description including dosage, strength, etc., an indication of the manufacturer, a unit price, a market status, a date the pharmaceutical was added to the list, and/or other information.

According to one embodiment, the prescription adjudication process 320 can read a database 345 of drug attributes for a plurality of pharmaceuticals in the plurality of categories identified above. For example, the database 345 can comprise the Medi-span database or similar database as known in the art. Maintaining the list 325 of pharmaceuticals can further comprise analyzing, by the prescription adjudication process 320, an available pharmaceutical based on the drug attributes of the plurality of pharmaceuticals in the database 345 and in a same category as the available pharmaceutical. The prescription adjudication process 320 can determine, based on analyzing the available pharmaceutical, whether the available pharmaceutical comprises a substitute pharmaceutical for at least one pharmaceutical of the plurality of pharmaceuticals in the same category as the available pharmaceutical and update the list of pharmaceuticals based on determining the available pharmaceutical comprises a substitute pharmaceutical for the at least one pharmaceutical of the plurality of pharmaceuticals in the same category as the available pharmaceutical.

When the prescription adjudication process 320 receives the prescription information 315 with the code identifying the prescribed pharmaceutical, the prescription adjudication process 320 can determine whether the prescribed pharmaceutical is an identified inefficiency based on the code identifying a prescribed pharmaceutical and the codes 335 identifying the pharmaceuticals in the list 325 of pharmaceuticals. In response to determining the prescribed pharmaceutical is an identified inefficiency, the prescription adjudication process 320 can provide, to the pharmacy system 310, a message 350 comprising instructions related to the prescribed pharmaceutical and medically or price-driven recommendations for decision support on the prescribed pharmaceutical based on the identified substitute of the pharmaceutical in the list 325 of pharmaceuticals. Additionally, or alternatively, the prescription adjudication process 320 can provide, to the adjuster system 360, recommendations and messages 355 related to the prescribed pharmaceutical based on the identified substitute of the pharmaceutical in the list 325 of pharmaceuticals. For example, the adjuster system 360 can comprise a system used by a third-party payor to review claims for payment.

The prescription adjudication process 320 can, in response to determining the prescribed pharmaceutical is not an identified inefficiency, determine whether the prescribed pharmaceutical comprises an outlier from substitute pharmaceuticals in a same category of pharmaceuticals as the prescribed pharmaceutical. The prescription adjudication process 320 can add the prescribed pharmaceuticals to the list 325 of pharmaceuticals in response to determining the prescribed pharmaceutical comprises an outlier. Determining whether the prescribed pharmaceutical comprises an outlier from substitute pharmaceuticals in a same category of pharmaceuticals as the prescribed pharmaceutical can comprise applying an interquartile range method to a price of the prescribed pharmaceutical and prices of the substitute pharmaceuticals in the same category of pharmaceuticals as the prescribed pharmaceutical. Determining whether the prescribed pharmaceutical comprises an outlier from substitute pharmaceuticals in a same category of pharmaceuticals as the prescribed pharmaceutical can be further based on a prescribed dosage of the prescribed pharmaceutical.

In this way, embodiments identify medications at the National Drug Code level that do not provide clinical value over more cost-effective, yet equally efficacious therapeutic alternatives. Embodiments identify price outliers to generate a list 325 which can be used as an exclusion drug list and insight tool for identifying price outlier medications for appropriate Fraud, Waste and Abuse (FWA) mitigation. Embodiments facilitate the identification of drugs having a higher cost than similar medications within the same category, without a corresponding increase in clinical value.

Embodiments categorize medications into specific groups such as compound Private-Label Topical Analgesics (PLTAs), topicals, fixed combos, select oral agents, and identifies high-priced outliers. This categorization aids in systematically identifying overpriced medications and allows for customizable downstream clinical interventions.

To enhance the identification process, embodiments can integrate with Medi-span or other databases. This integration allows for the accurate detection of compound kits or combo packs, for example, which are often individual components or alternative options.

Embodiments can also include a feature for analyzing new GPIs and potential inclusion in the targeted list of medications. This allows the list to remain up-to-date and reflective pharmaceutical landscape.

For example, utilizing the interquartile range method, embodiments can analyze the pricing data of an ibuprofen 800 mg tablet is priced at $12, whereas a similar ibuprofen 800 mg tablet is available at $0.12. The higher priced tablet can be identified as a price outlier for further review and potential exclusion from procurement lists.

Through integration with Medi-span and/or other databases and use of product descriptions, embodiments can identify compound kits and combo packs. By highlighting these options, healthcare providers can select a cost-effective, individual medications or alternative combinations. An example would be a combination of lidocaine adhesive sheet kit priced at $3200 versus the products individually being available for less than $100 for a 1-month supply.

Embodiments can assess PLTAs, which frequently combine Over-The-Counter (OTC) pharmaceuticals available at a fraction of the cost. By identifying these PLTAs, embodiments help healthcare providers avoid manufacturer markups.

As new medications enter the market, embodiments can evaluate their Generic Product Identifiers for relevance and cost-effectiveness. This allows inclusion of the most therapeutically and economically viable medications in a provider's or payor's intended formulary. Embodiments further allow decision makers to make informed, cost-effective medication choices based on clinical value.

As noted, embodiments can detecting price outliers based on the interquartile range and objectively flag medications that are priced significantly higher than their therapeutic equivalents. This goes beyond simple price comparisons, incorporating statistical analysis to identify anomalies which could indicate overcharging or unjustifiable pricing strategies by manufacturers.

According to one embodiment, drugs can be analyzed at the GPI-12 level to identify dose strengths and dosage forms at the NDC level for therapeutic differentiator. This granular level of analysis allows comparisons and recommendations that are accurate and directly addresses the issue of high-priced topical agents, combination drugs, and the unnecessary use of higher-priced drugs when more cost-effective alternatives exist. This level of detail provides for identifying not just broad categories of potential opportunities but also to reduce costs without compromising patient care.

In the context of workers' compensation and Pharmacy Benefit Managers (PBMs), the focus on eliminating wasteful and low-value medication is especially pertinent. An ability to identify potential FWA is critical, as these sectors are vulnerable to such activities due to the complex nature of billing and reimbursement. By automating the detection of overpriced or outlier medications, embodiments can directly contribute to more sustainable and financially responsible pharmacy benefit programs.

The automation of NDC data analysis significantly reduces the time and effort traditionally required to manually control spending. This efficiency not only cuts operational costs but also allows for more rapid adjustments to formulary strategies, ensuring that employers' pharmacy benefit plans are continuously optimized for both cost and clinical efficacy.

While embodiments are described as identifying outliers based on the interquartile range alternatives are contemplated and considered to be within the scope of the present disclosure. For example, machine learning and artificial intelligence can be utilized in one embodiment to provide a more dynamic and predictive analysis of drug pricing and utilization patterns. Such embodiment could include consideration of variables, including historical pricing data, market trends, and even predictive modeling for future price changes using GPI, GCN (Generic Code Number), or NDC data as elements. Embodiments can comprise training models on vast datasets to recognize patterns indicating possible Fraud, Waste, and Abuse (FWA) or to predict which medications are likely to become overpriced relative to their substitutes. Such automation could significantly reduce the manual effort required in the current PBM processes, making the system scalable.

While embodiments are described above as being useful to the workers' compensation industry and pharmacy benefit managers, embodiments can be customized for various healthcare sectors. Such implementations can be adapted for different patient demographics (considering age, sex, disease states, etc.) or healthcare settings networks, outpatient clinics, or specialty pharmacies). When applied to a different healthcare sector, cost savings achieved can be redirected towards more valuable healthcare initiatives or used to lower the cost burden on patients via reduced adherence and outcomes.

According to one embodiment, patient-centric data can be integrated into the formulary design process. Such data can include, but is not limited to, outcomes, adherence rates, and demographic information, which could be used to offer more personalized recommendations. This approach could improve patient care by ensuring that formulary decisions are aligned with patient-specific needs and outcomes.

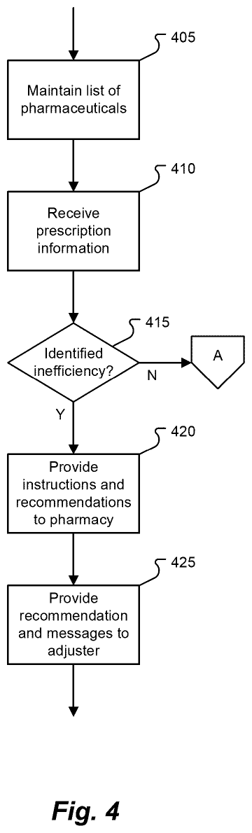

FIG. 4 is a flowchart illustrating an exemplary process for identifying and controlling inefficiencies in pharmaceutical utilization according to one embodiment of the present disclosure. As illustrated in this example, identifying and controlling inefficiencies in pharmaceutical utilization can comprise maintaining 405 a list of pharmaceuticals comprising a plurality of categories. Each category can comprise a plurality of pharmaceuticals, each pharmaceutical can be identified by a code, and each pharmaceutical in the list of pharmaceuticals can comprise an identified inefficiency. The list of pharmaceuticals can further comprise an identified substitute for each pharmaceutical in the list of pharmaceuticals.

Information comprising a code identifying a prescribed pharmaceutical can be received 410 from a pharmacy system and a determination 415 can be made as to whether the prescribed pharmaceutical is an identified inefficiency based on the code identifying a prescribed pharmaceutical and the codes identifying the pharmaceuticals in the list of pharmaceuticals. In response to determining 415 the prescribed pharmaceutical is an identified inefficiency, instructions related to the prescribed pharmaceutical and medically or price-driven recommendations for decision support on the prescribed pharmaceutical based on the identified substitute of the pharmaceutical in the list of pharmaceuticals having a code matching the code identifying the prescribed pharmaceutical can be provided 420 to the pharmacy system. Additionally, or alternatively, recommendations and messages related to the prescribed pharmaceutical based on the identified substitute of the pharmaceutical in the list of pharmaceuticals having a code matching the code identifying the prescribed pharmaceutical can be provided 425 to an adjuster system.

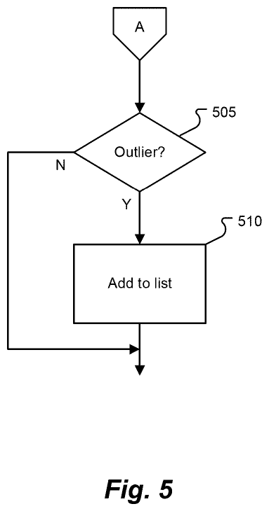

FIG. 5 is a flowchart illustrating additional details of an exemplary process for identifying and controlling inefficiencies in pharmaceutical utilization according to one embodiment of the present disclosure. As illustrated in this example, in response to determining 415 the prescribed pharmaceutical is not an identified inefficiency based on the code identifying a prescribed pharmaceutical and the codes identifying the pharmaceuticals in the list of pharmaceuticals, a further determination 505 can be made as to whether the prescribed pharmaceutical comprises an outlier from substitute pharmaceuticals in a same category of pharmaceuticals as the prescribed pharmaceutical. Determining 505 whether the prescribed pharmaceutical comprises an outlier from substitute pharmaceuticals in a same category of pharmaceuticals as the prescribed pharmaceutical can comprise applying an interquartile range method to a price of the prescribed pharmaceutical and prices of the substitute pharmaceuticals in the same category of pharmaceuticals as the prescribed pharmaceutical. Determining 505 whether the prescribed pharmaceutical comprises an outlier from substitute pharmaceuticals in a same category of pharmaceuticals as the prescribed pharmaceutical is further based on a prescribed dosage of the prescribed pharmaceutical. Maintaining 405 the list of pharmaceuticals can comprise adding 510 the prescribed pharmaceuticals to the list of pharmaceuticals in response to determining 505 the prescribed pharmaceutical comprises an outlier from the substitute pharmaceuticals in the same category of pharmaceuticals as the prescribed pharmaceutical.

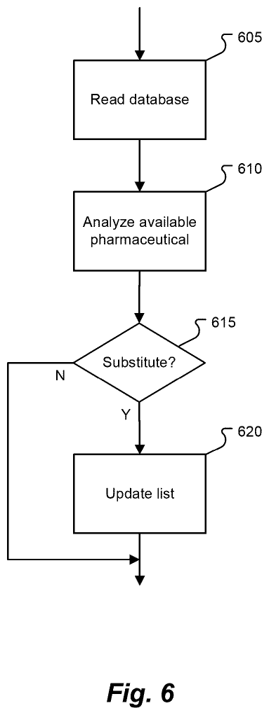

FIG. 6 is a flowchart illustrating further details of an exemplary process for identifying and controlling inefficiencies in pharmaceutical utilization according to one embodiment of the present disclosure. As illustrated in this example, maintaining 405 the list of pharmaceuticals can further comprise reading 605 a database of drug attributes for a plurality of pharmaceuticals in a plurality of categories. An available pharmaceutical can be analyzed 610 based on the drug attributes of the plurality of pharmaceuticals in a same category as the available pharmaceutical. A determination 615 can be made, based on analyzing the available pharmaceutical, whether the available pharmaceutical comprises a substitute pharmaceutical for at least one pharmaceutical of the plurality of pharmaceuticals in the same category as the available pharmaceutical. The list of pharmaceuticals can then be updated 620 based on determining 615 the available pharmaceutical comprises a substitute pharmaceutical for the at least one pharmaceutical of the plurality of pharmaceuticals in the same category as the available pharmaceutical.

The database of drug attributes can further comprise information identifying pharmaceutical compound kits, combinations packs, private label topical analgesics, prescription topicals, fixed combinations, oral agents and test supplies. In such cases, determining 415 whether the prescribed pharmaceutical is an identified inefficiency can further comprise determining whether the prescribed pharmaceutical is part of a pharmaceutical compound kits, combinations packs, private label topical analgesics, prescription topicals, fixed combinations, oral agents or test supplies pack based on the information identifying pharmaceutical compound kits, combinations packs, private label topical analgesics, prescription topicals, fixed combinations, oral agents and test supplies from the database of drug attributes.

The present disclosure, in various aspects, embodiments, and/or configurations, includes components, methods, processes, systems, and/or apparatus substantially as depicted and described herein, including various aspects, embodiments, configurations embodiments, sub-combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for identifying and controlling inefficiencies in pharmaceutical utilization, the method comprising:
maintaining, by a processor of an adjudication system, a list of pharmaceuticals comprising a plurality of categories, wherein each category comprises a plurality of pharmaceuticals, each pharmaceutical being identified by a code, wherein each pharmaceutical in the list of pharmaceuticals comprises an identified inefficiency, and wherein the list of pharmaceuticals further comprises an identified substitute for each pharmaceutical in the list of pharmaceuticals;
receiving, by the processor of the adjudication system, from a pharmacy system, information comprising a code identifying a prescribed pharmaceutical;
determining, by the processor of the adjudication system, whether the prescribed pharmaceutical is an identified inefficiency based on the code identifying a prescribed pharmaceutical and the codes identifying the pharmaceuticals in the list of pharmaceuticals; and
in response to determining the prescribed pharmaceutical is an identified inefficiency, providing, by the processor of the adjudication system, to the pharmacy system, instructions related to the prescribed pharmaceutical and medically or price-driven recommendations for decision support on the prescribed pharmaceutical based on the identified substitute of the pharmaceutical in the list of pharmaceuticals having a code matching the code identifying the prescribed pharmaceutical.

2. The method of claim 1, further comprising providing, by the processor of the adjudication system, to an adjuster system, recommendations and messages related to the prescribed pharmaceutical based on the identified substitute of the pharmaceutical in the list of pharmaceuticals having a code matching the code identifying the prescribed pharmaceutical.

3. The method of claim 1, further comprising in response to determining the prescribed pharmaceutical is not an identified inefficiency based on the code identifying a prescribed pharmaceutical and the codes identifying the pharmaceuticals in the list of pharmaceuticals, determining, by the processor of the adjudication system, whether the prescribed pharmaceutical comprises an outlier from substitute pharmaceuticals in a same category of pharmaceuticals as the prescribed pharmaceutical.

4. The method of claim 3, wherein maintaining the list of pharmaceuticals comprises adding the prescribed pharmaceuticals to the list of pharmaceuticals in response to determining the prescribed pharmaceutical comprises an outlier from the substitute pharmaceuticals in the same category of pharmaceuticals as the prescribed pharmaceutical.

5. The method of claim 3, wherein determining whether the prescribed pharmaceutical comprises an outlier from substitute pharmaceuticals in a same category of pharmaceuticals as the prescribed pharmaceutical comprises applying an interquartile range method to a price of the prescribed pharmaceutical and prices of the substitute pharmaceuticals in the same category of pharmaceuticals as the prescribed pharmaceutical.

6. The method of claim 3, wherein determining whether the prescribed pharmaceutical comprises an outlier from substitute pharmaceuticals in a same category of pharmaceuticals as the prescribed pharmaceutical is further based on a prescribed dosage of the prescribed pharmaceutical.

7. The method of claim 1, further comprising reading a database of drug attributes for a plurality of pharmaceuticals in a plurality of categories.

8. The method of claim 7, wherein maintaining the list of pharmaceuticals further comprises:
analyzing an available pharmaceutical based on the drug attributes of the plurality of pharmaceuticals in a same category as the available pharmaceutical;
determining, based on analyzing the available pharmaceutical, whether the available pharmaceutical comprises a substitute pharmaceutical for at least one pharmaceutical of the plurality of pharmaceuticals in the same category as the available pharmaceutical; and
updating the list of pharmaceuticals based on determining the available pharmaceutical comprises a substitute pharmaceutical for the at least one pharmaceutical of the plurality of pharmaceuticals in the same category as the available pharmaceutical.

9. The method of claim 7, wherein the database of drug attributes further comprises information identifying pharmaceutical compound kits, combinations packs, private label topical analgesics, prescription topicals, fixed combinations, oral agents and test supplies, and wherein determining whether the prescribed pharmaceutical is an identified inefficiency further comprises determining whether the prescribed pharmaceutical is part of a pharmaceutical compound kits, combinations packs, private label topical analgesics, prescription topicals, fixed combinations, oral agents or test supplies pack based on the information identifying pharmaceutical compound kits, combinations packs, private label topical analgesics, prescription topicals, fixed combinations, oral agents and test supplies from the database of drug attributes.

10. A system comprising:
a processor; and
a memory coupled with and readable by the processor and storing therein a set of instructions which, when executed by the processor, causes the processor to:
maintain a list of pharmaceuticals comprising a plurality of categories, wherein each category comprises a plurality of pharmaceuticals, each pharmaceutical being identified by a code, wherein each pharmaceutical in the list of pharmaceuticals comprises an identified inefficiency, and wherein the list of pharmaceuticals further comprises an identified substitute for each pharmaceutical in the list of pharmaceuticals;
receive, from a pharmacy system, information comprising a code identifying a prescribed pharmaceutical;
determine whether the prescribed pharmaceutical is an identified inefficiency based on the code identifying a prescribed pharmaceutical and the codes identifying the pharmaceuticals in the list of pharmaceuticals; and
in response to determining the prescribed pharmaceutical is an identified inefficiency, provide, to the pharmacy system, instructions related to the prescribed pharmaceutical and medically or price-driven recommendations for decision support on the prescribed pharmaceutical based on the identified substitute of the pharmaceutical in the list of pharmaceuticals having a code matching the code identifying the prescribed pharmaceutical.

11. The system of claim 10, wherein the instructions further cause the processor to provide, to an adjuster system, recommendations and messages related to the prescribed pharmaceutical based on the identified substitute of the pharmaceutical in the list of pharmaceuticals having a code matching the code identifying the prescribed pharmaceutical.

12. The system of claim 10, wherein the instructions further cause the processor to, in response to determining the prescribed pharmaceutical is not an identified inefficiency based on the code identifying a prescribed pharmaceutical and the codes identifying the pharmaceuticals in the list of pharmaceuticals, determine whether the prescribed pharmaceutical comprises an outlier from substitute pharmaceuticals in a same category of pharmaceuticals as the prescribed pharmaceutical.

13. The system of claim 12, wherein maintaining the list of pharmaceuticals comprises adding the prescribed pharmaceuticals to the list of pharmaceuticals in response to determining the prescribed pharmaceutical comprises an outlier from the substitute pharmaceuticals in the same category of pharmaceuticals as the prescribed pharmaceutical.

14. The system of claim 12, wherein determining whether the prescribed pharmaceutical comprises an outlier from substitute pharmaceuticals in a same category of pharmaceuticals as the prescribed pharmaceutical comprises applying an interquartile range method to a price of the prescribed pharmaceutical and prices of the substitute pharmaceuticals in the same category of pharmaceuticals as the prescribed pharmaceutical.

15. The system of claim 12, wherein determining whether the prescribed pharmaceutical comprises an outlier from substitute pharmaceuticals in a same category of pharmaceuticals as the prescribed pharmaceutical is further based on a prescribed dosage of the prescribed pharmaceutical.

16. The system of claim 10, wherein the instructions further cause the processor to read a database of drug attributes for a plurality of pharmaceuticals in a plurality of categories.

17. The system of claim 16, wherein maintaining the list of pharmaceuticals further comprises:
analyzing an available pharmaceutical based on the drug attributes of the plurality of pharmaceuticals in a same category as the available pharmaceutical;
determining, based on analyzing the available pharmaceutical, whether the available pharmaceutical comprises a substitute pharmaceutical for at least one pharmaceutical of the plurality of pharmaceuticals in the same category as the available pharmaceutical; and
updating the list of pharmaceuticals based on determining the available pharmaceutical comprises a substitute pharmaceutical for the at least one pharmaceutical of the plurality of pharmaceuticals in the same category as the available pharmaceutical.

18. The system of claim 16, wherein the database of drug attributes further comprises information identifying pharmaceutical compound kits, combinations packs, private label topical analgesics, prescription topicals, fixed combinations, oral agents and test supplies, and wherein determining whether the prescribed pharmaceutical is an identified inefficiency further comprises determining whether the prescribed pharmaceutical is part of a pharmaceutical compound kits, combinations packs, private label topical analgesics, prescription topicals, fixed combinations, oral agents or test supplies pack based on the information identifying pharmaceutical compound kits, combinations packs, private label topical analgesics, prescription topicals, fixed combinations, oral agents and test supplies from the database of drug attributes.

19. A non-transitory, computer-readable medium comprising a set of instructions stored therein which, when executed by a processor, causes the processor to:
maintain a list of pharmaceuticals comprising a plurality of categories, wherein each category comprises a plurality of pharmaceuticals, each pharmaceutical being identified by a code, wherein each pharmaceutical in the list of pharmaceuticals comprises an identified inefficiency, and wherein the list of pharmaceuticals further comprises an identified substitute for each pharmaceutical in the list of pharmaceuticals;
receive, from a pharmacy system, information comprising a code identifying a prescribed pharmaceutical;
determine whether the prescribed pharmaceutical is an identified inefficiency based on the code identifying a prescribed pharmaceutical and the codes identifying the pharmaceuticals in the list of pharmaceuticals; and
in response to determining the prescribed pharmaceutical is an identified inefficiency, provide, to the pharmacy system, instructions related to the prescribed pharmaceutical and medically or price-driven recommendations for decision support on the prescribed pharmaceutical based on the identified substitute of the pharmaceutical in the list of pharmaceuticals having a code matching the code identifying the prescribed pharmaceutical.

20. The non-transitory, computer-readable medium of claim 19, wherein the instructions further cause the processor to provide, to an adjuster system, recommendations and messages related to the prescribed pharmaceutical based on the identified substitute of the pharmaceutical in the list of pharmaceuticals having a code matching the code identifying the prescribed pharmaceutical.

21. The non-transitory, computer-readable medium of claim 19, wherein the instructions further cause the processor to, in response to determining the prescribed pharmaceutical is not an identified inefficiency based on the code identifying a prescribed pharmaceutical and the codes identifying the pharmaceuticals in the list of pharmaceuticals, determine whether the prescribed pharmaceutical comprises an outlier from substitute pharmaceuticals in a same category of pharmaceuticals as the prescribed pharmaceutical.

22. The non-transitory, computer-readable medium of claim 21, wherein maintaining the list of pharmaceuticals comprises adding the prescribed pharmaceuticals to the list of pharmaceuticals in response to determining the prescribed pharmaceutical comprises an outlier from the substitute pharmaceuticals in the same category of pharmaceuticals as the prescribed pharmaceutical.

23. The non-transitory, computer-readable medium of claim 21, wherein determining whether the prescribed pharmaceutical comprises an outlier from substitute pharmaceuticals in a same category of pharmaceuticals as the prescribed pharmaceutical comprises applying an interquartile range method to a price of the prescribed pharmaceutical and prices of the substitute pharmaceuticals in the same category of pharmaceuticals as the prescribed pharmaceutical.

24. The non-transitory, computer-readable medium of claim 21, wherein determining whether the prescribed pharmaceutical comprises an outlier from substitute pharmaceuticals in a same category of pharmaceuticals as the prescribed pharmaceutical is further based on a prescribed dosage of the prescribed pharmaceutical.

25. The non-transitory, computer-readable medium of claim 19, wherein the instructions further cause the processor to read a database of drug attributes for a plurality of pharmaceuticals in a plurality of categories.

26. The non-transitory, computer-readable medium of claim 25, wherein maintaining the list of pharmaceuticals further comprises:
analyzing an available pharmaceutical based on the drug attributes of the plurality of pharmaceuticals in a same category as the available pharmaceutical;
determining, based on analyzing the available pharmaceutical, whether the available pharmaceutical comprises a substitute pharmaceutical for at least one pharmaceutical of the plurality of pharmaceuticals in the same category as the available pharmaceutical; and
updating the list of pharmaceuticals based on determining the available pharmaceutical comprises a substitute pharmaceutical for the at least one pharmaceutical of the plurality of pharmaceuticals in the same category as the available pharmaceutical.

27. The non-transitory, computer-readable medium of claim 25, wherein the database of drug attributes further comprises information identifying pharmaceutical compound kits, combinations packs, private label topical analgesics, prescription topicals, fixed combinations, oral agents and test supplies, and wherein determining whether the prescribed pharmaceutical is an identified inefficiency further comprises determining whether the prescribed pharmaceutical is part of a pharmaceutical compound kits, combinations packs, private label topical analgesics, prescription topicals, fixed combinations, oral agents or test supplies pack based on the information identifying pharmaceutical compound kits, combinations packs, private label topical analgesics, prescription topicals, fixed combinations, oral agents and test supplies from the database of drug attributes.
